(19) United States
(12) Patent Application Publication
(10) Pub. No.: US 2011/0217239 A1
(43) Pub. Date: Sep. 8, 2011

(54) SYSTEM AND METHOD OF EVALUATING A PROTEIN OF INTEREST ON TUMOR GROWTH INHIBITION WHILE FOLLOWING THE TUMOR IN VIVO OR IN VITRO

(75) Inventors: Yi-Jang LEE, Taipei City (TW); Cheng-Han TSAI, Taipei City (TW); Liang-Ting LIN, Taipei City (TW)

(73) Assignees: Yi-Jang LEE, Taipei City (TW); Cheng-Han TSAI, Taipei City (TW); Liang-Ting LIN, Taipei City (TW)

(21) Appl. No.: 12/717,143

(22) Filed: Mar. 4, 2010

(51) Int. Cl. A61K 49/00; A61K 31/7088; C12N 5/09; C12N 5/071; A61P 35/00

(52) U.S. Cl. 424/9.2; 514/44 R; 435/325; 435/366

(57) ABSTRACT

Disclosed herein are systems and methods for evaluating a protein of interest on growth inhibition of a tumor while following the tumor in vivo or in vitro through a co-expressed visible marker. The present disclosure provides an expression composition for stably transforming cells, preferably tumor cells. The expression composition includes two vectors respectively include polynucleotides encoding a protein of interest and a visible marker, wherein each polynucleotide is operably linked to a tet-on system such that the protein of interest and the visible marker are co-expressed upon activation of the tet-on system. The present disclosure also provides stably transfected cell lines, which may be used to access real-time biological processes, including tumor cell proliferation.

### TECHNICAL FIELD

This disclosure in general relates to systems and methods for evaluating a therapeutic protein candidate on a tumor while simultaneously tracking and/or monitoring the growth of the tumor in vivo or in vitro. More particularly, this disclosure relates to expression systems and methods for simultaneously retarding and monitoring the growth of a tumor in a live subject.

### BACKGROUND ART

### Description of Related Art

A key feature in understanding a biological event is the ability to monitor biological event in vivo. One area of medicine that would benefit from the ability of monitoring real-time biological event is the field of tumor research. Despite significant improvements in developing treatments for various cancers, most of the researches remain in vitro studies. Some animal models have been developed for the study of a wide range of diseases and provide appropriate vehicles for drug validation or evaluation. For example, transgenic mice containing suitable elements have been developed for the studies of diabetes, obesity, cardiovascular diseases and etc. However, transgenic animals produced by current methods usually lack of suitable means (i.e., non-invasive means) of following the expression of the interested genes, and would require killing the animals and preparing the histological or immunological sections for identification. Therefore, it would have been a distinct advantage to have available an improved means to monitor a protein of interest, such as a therapeutic protein of a tumor in vivo, e.g., to easily and rapidly identify proliferating tumor cells, and to enable examination of the effects of the therapeutic protein simultaneously.

This invention address such need by providing an improved means for evaluating the effects of a therapeutic protein candidate on a tumor, while simultaneously following the growth of the tumor in vivo or in vitro.

### SUMMARY

As embodied and broadly described herein, disclosure herein features a novel means for evaluating the therapeutic effects of a therapeutic protein candidate on a tumor, while simultaneously following the growth of the tumor in vivo.

Therefore, it is the objective of the present disclosure to provide suitable system and/or method for evaluating the therapeutic effects of a therapeutic protein candidate on a tumor, while simultaneously following the growth of the tumor in vivo. The system may be a modified vertebrate containing tumor cells. The tumor cells are modified by an expression composition to express a therapeutic protein candidate and a visible marker simultaneously, such that the tumor are tracked and/or monitored by the visible marker while the growth of the tumor is suppressed or retarded by the expressed therapeutic protein candidate.

According to specific embodiments of the present disclosure, an expression composition is provided. The expression composition includes a first vector, which comprises a first polynucleotide encoding a therapeutic protein candidate; and a second vector, which comprises a second polynucleotide encoding a visible marker that is capable of emitting a luminescence or fluorescence signal; wherein the first and second polynucleotides of the first and second vectors are respectively regulated by a tet-on system operably linked to the respective first and second polynucleotides such that the therapeutic protein candidate and the visible marker are simultaneously expressed upon activating the tet-on system. According to specific examples of the present embodiment, the tet-on system is activated by doxycyclin, which in turn activates downstream gene expression. The first and second vectors are respectively replicable in normal or cancerous cells, and are respectively selected from the group consisting of an adenovirus, an adenovirus associated virus, a retrovirus and a lentivirus. According to specific example of the present disclosure, the therapeutic protein candidate is cofilin, and the visible marker is selected from the group consisting of luciferase, green fluorescence protein (GFP), yellow fluorescence protein (YFP), red fluorescence protein (RFP), orange fluorescence protein (OFP), cyan fluorescence protein (CFP), and UV-excitable green fluorescence protein (UV-GFP).

According to another embodiment of the present disclosure, a cell stably transfected with the expression composition of the present disclosure is provided. According to examples of this embodiment, the cell may be a tumor cell that is selected from the group consisting of melanoma cell, ovary cancer cell, lung cancer cell, breast cancer cell and prostate cancer cell. In one specific embodiment, the cell is a lung cancer cell.

According to still another embodiment of the present disclosure, a method for evaluating a therapeutic protein candidate on the growth of a tumor while simultaneously following the tumor in a live subject is provided. The method includes steps of: injecting the subject with the cell stably transfected with the composition of the present disclosure; administering to the subject an effective amount of doxycyclin to activate the tet-on system and thereby activating the expression of the therapeutic protein candidate and the visible marker simultaneously; and monitoring the growth of the tumor by the luminescence or fluorescence signal emitted from the visible marker; wherein the luminescence or fluorescence signal first increases with the growth of the tumor and subsequently diminishes as the growth of the tumor is retarded by the expressed therapeutic protein candidate. According to specific examples of this disclosure, the doxycyclin is administered in an amount of about 1 to 1000 μg/Kg, preferably about 100 to 800 μg/Kg, more preferably about 400 to 600 μg/Kg, and most preferably about 500 μg/Kg. The tumor is selected from the group consisting of melanoma, ovary cancer, lung cancer, breast cancer cell and prostate cancer. The therapeutic protein candidate is cofilin. The subject is a non-human mammal, and preferably is a mouse.

The details of one or more embodiments of the invention are set forth in the accompanying description below. Other features and advantages of the invention will be apparent from the detail descriptions, and from claims.

It is to be understood that both the foregoing general description and the following detailed description are by examples, and are intended to provide further explanation of the invention as claimed.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

The accompanying drawings are included to provide a further understanding of the invention, and are incorporated in and constitute a part of this specification. The drawings illustrate embodiments of the invention and, together with the description, serve to explain the principles of the invention. In the drawings,

FIG. 1A illustrates the expression of cofilin in HCOXP-tk-Luc cells of Example 1.3 treated with various concentration of doxycyclin in accordance with one preferred embodiment of this invention;

FIG. 1B illustrates the luminance measured at different time points from HCOXP-tk-Luc cells of Example 1.3 in the presence or absence of doxycyclin in accordance with one preferred embodiment of this invention;

FIG. 1C illustrates the co-expression of thymidine kinase and cofilin in HCOXP-tk-EGFP cells of Example 1.3 treated with doxycyclin (1 μg/ml) in accordance with one preferred embodiment of this invention;

FIGS. 2A and 2B illustrate the tumor size measured in mice having xenografted HCOXP-tk cells of example 1.2 and HCOXP-tk-luc cells of example 1.3, respectively in the presence or absence of doxycyclin treatment in accordance with one preferred embodiment of this invention;

FIGS. 2C and 2D illustrate the luminance measured in mice having xenografted HCOXP-tk cells of example 1.2 and HCOXP-tk-luc cells of example 1.3, respectively in the presence or absence of doxycyclin treatment in accordance with one preferred embodiment of this invention;

FIG. 2E are IVIS images taken from mice having xenografted HCOXP-tk cells of example 1.2 and HCOXP-tk-luc cells of example 1.3 on their left and right feet, respectively in the presence or absence of doxycyclin treatment in accordance with one preferred embodiment of this invention, in which Dox (1 μg/ml) was added to the drinking water;

FIG. 3A illustrates the luminance intensity of a mouse having xenografted HCOXP-tk-luc cells of example 1.3 in the presence or absence of doxycyclin to treatment in accordance with one preferred embodiment of this invention;

FIG. 3B are IVIS images taken from the same mouse of FIG. 3A from week 1 to 5, respectively, with the arrow indicated the site of the tumor;

FIG. 4A illustrates the fluorescence intensity of a mouse having xenografted HCOXP-tk-EGFP cells of example 1.3 in the presence or absence of doxycyclin treatment in accordance with one preferred embodiment of this invention;

FIG. 4B are IVIS images taken from the same mouse of FIG. 4A from week 1 to 5, respectively, with the arrow indicated the site of the tumor;

FIGS. 5A and 5B illustrate the images taken by microPET and gamma camera, respectively from mice having xenografted tumor cells in the presence or absence of doxycyclin treatment in accordance with one preferred embodiment of this invention; and

FIGS. 5C and 5D respectively illustrates the microPET immagies and immunostaining analysis of mice having xenografted tumor cells in the presence or absence of doxycyclin treatment in accordance with one preferred embodiment of this invention.

### DETAIL DESCRIPTION OF THE INVENTION

### Definitions

The following terms have been used to describe the present invention.

As used herein, the term “polynucleotide” refers to polymeric form of nucleotides in any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA, transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers.

The term “encode” herein refers to a polynucleotide or a nucleic acid sequence which codes for a polypeptide sequence, or a portion thereof that contains amino acid sequence of at least 3 to 5 amino acids, more preferably at least 8 to 10 amino acids, and even more preferably at least 15 to 20 amino acids from a polypeptide encoded by the polynucleotide or nucleic acid sequence. Also encompasses are polypeptide sequences which are immunological identifiable within a polypeptide encoded by the sequence.

The term “operably linked” refers to an arrangement of elements wherein the components so described are configured to perform their usual functions. Thus, a tet-on system operably linked to a polynucleotide is capable of effecting transcription of the polynucleotide when a proper activator, such as doxycyclin, is present to turn on the tet-on system.

The term “vector” refers to a vehicle, preferably a nucleic acid molecule that can transport a desired polynucleotide sequence into a cell. Non-limiting examples of vectors include a plasmid, single or double strand phage, a single or double stranded RNA or DNA viral vector, or artificial chromosome, such as bacterial artificial chromosome (BAC), plant artificial chromosome (PAC), yeast artificial chromosome (YAC), mammalian artificial chromosome (MAC) or human artificial chromosome (HAC). A vector can be maintained in a host cell as an extrachromosomal element where it replicates and produces additional copies of the desired polynucleotide, or can integrate into the host cell genome.

The term “non-human mammal” as used herein including, without limitation, farm animals such as cattle, sheep, pigs, goats and horses or domestic mammals such as dogs and cats; laboratory animals including rodents such as mice, rats and guinea pigs; birds, including domestic, wild and game birds such as chickens, turkeys and other gallinaceous birds, ducks, geese, and the like; vertebrates, such as non-human primates, cows; amphibians; reptiles and etc. The term does not denote a particular age. Thus, both adult and newborn individuals are intended to be covered. In preferred embodiments, a non-human mammal useful in the method and/or system of the present disclosure is a rodent, and in more preferred embodiments, the animal is a mouse.

### An Overview of the Invention

The practices of this invention are hereinafter described in detail with respect to a model system for studying the effects of a therapeutic protein on growth inhibition of tumors. More particularly, the present disclosure relates to developing a model system for testing or evaluating a protein of interest on growth inhibition of a tumor while simultaneously following the tumor in vivo. Advantages is taken of the co-expression of a therapeutic protein candidate (e.g., a tumor growth inhibitor) as well as a visible marker (e.g., a luminance or fluorescence protein) that labels the tumor cells, such that the growth of tumor cells are suppressed and/or inhibited by the expressed therapeutic protein candidate while the growth of the tumor cells can be followed simultaneously by the co-expressed luminant or fluorescent protein.

In general, the model system of the present disclosure involves modifying a vertebrate, preferably a non-human mammal, so as to contain tumor cells, wherein the tumor cells have been modified to express a therapeutic protein candidate and a visible marker through a tet-on system respectively linked thereto. The tumor cells may arise from the cell lines of this invention, wherein the tumor cells have been modified by an expression composition to express a therapeutic protein candidate and a visible marker, simultaneously. Tumors can be formed in such vertebrate system by administering the transformed cells containing the expression composition of the present disclosure, and permitting the transformed cells to form tumors. Typically such administration is subcutaneous and the tumors are formed as solid tumors. The tumors thus formed can be implanted in any suitable host tissue and allow tumor to progress and develop. Since sufficient photon intensity may be achieved to observe the luminant or fluorescent proteins emitted from the co-expressed visible marker in the tumor cells, the tumor in the subject may be monitored or tracked in real-time, hence the therapeutic effects of the therapeutic protein candidate on tumor growth may be evaluated in real-time, by simply following the intensity change of the expressed luminant or fluorescent protein. As tumor cells grow, luminance or fluorescence intensity in tumor cells would increase accordingly, in the case when the expressed therapeutic protein candidate is effective in retarding, suppressing or inhibiting tumor growth, the luminance or fluorescence intensity in tumor cells would subsequently diminish as the tumor size shrinks due to growth inhibition conferred by the expressed therapeutic protein candidate. On the other hand, if the expressed therapeutic protein candidate does not produce satisfactory effects on growth inhibition, tumor size would continue to increase with time, and so is the luminance or fluorescence intensity in tumor cells.

### Generation of the Model System of the Invention

It is thus a first aspect of this invention to provide a composition for modifying a tumor cell. The composition comprises a first and second vectors, which respectively comprise a first and second polynucleotides that respectively encode a therapeutic protein candidate and a visible marker, wherein the first and second polynucleotides are respectively regulated by a tet-on system operably linked to the first and second polynucleotides for respectively regulating the expression of the therapeutic protein candidate and the visible marker, such that the therapeutic protein candidate and the visible marker are simultaneously expressed upon activating the tet-on system.

The concept and principle of a Tet regulatory system was first described by Gossen and Bujard (Proc. Natl. Acad. Sci. USA (1992) 89(12): 5547-5551). In the tet-off system, gene expression is turned on when tetracycline (Tc) or doxycyclin (Dox; a Tc derivative) is removed from the culture medium. In contrast, gene expression is turned on in the tet-on system by the addition of Dox, and the tet-on system is responsive only to Dox, not Tc. Further, both systems permit gene expression to be tightly regulated in response to varying concentrations of Tc or Dox. In the present disclosure, in order to activate two proteins in a controlled manner (e.g., simultaneously), two tet-on systems are respectively linked to the first and second vectors so as to activate the expression of the therapeutic protein candidate and the visible marker, simultaneously, upon Dox treatment.

The vectors utilized in a preferred embodiment of the present disclosure are those which are described in the Examples section and the techniques necessary for constructing an expression vector for practicing this invention are readily available and well known. Specifically, vectors useful in practicing this invention are those comprising a tet-on system, such vectors are available commercially form various suppliers, and any appropriate gene of interest may be isolated from the organism in which it is found and ligated into the vector in a manner so that its expression is controlled by the operably linked tet-on system. For isolating the gene of interest, a DNA or cDNA library can be constructed and screened for the presence of the target gene. Methods of constructing and screening such libraries are well known in the arts and kits for performing the construction and screening steps are commercially available. Once isolated, the gene of interest can be directly cloned into the vector, or if necessary, be modified to facilitate the subsequent cloning steps. General methods of isolating and cloning a target gene are set forth in Sambrook et al., “Molecular Cloning, a Laboratory Mannual,” Cold Spring Harbor Laboratory Press (1989). Another way of obtaining a gene of interest is to amplify the nucleic acid corresponding to the gene of interest from the nucleic acids found within a host organism and clones the amplified gene in the vector. Further, it will be readily comprehended by those of skill in the art that the vectors described herein may be modified in several ways within the scope of this invention.

The therapeutic protein candidate may be any protein of interest, preferably the one having therapeutic effects on growth inhibition, cell necrosis and/or anti-proliferation. Examples of therapeutic protein include, but are not limited to, actin depolymerization factor (ADF), cofilin family protein such as cofilin, CFL-1, CFL-2, destrin, UNC-60A, UNC-60B, depactin, actophorin, coactosin, twinstar, TgADF XAC1 and XAC2. According to one specific embodiment of the present invention, the therapeutic protein candidate is cofilin.

Visible markers that are useful in the practice of this invention include luminescence or fluorescence generating proteins. Suitable luminescence generating protein includes both lux genes (prokaryotic genes encoding a luciferase activity) and luc genes (eukaryotic genes encoding a luciferase activity). A variety of luciferase encoding genes has been identified (see U.S. Pat. Nos. 5,670,356; 5,618,722; 5,650,289 and 5,229,285). Further, it is well known that luciferase catalyzes a reaction using luciferin as a luminescent substrate to produce luminescence. Suitable fluorescence generating protein that may be used to label tumor and follow its growth in vivo includes, but is not limited to, green fluorescence protein (GFP) such as enhanced green fluorescence protein (EGFP) from Aequorea Victoria; yellow fluorescence protein (YFP) such as Citrine, Venus, and Ypet; red fluorescence protein (RFP) such as coral red fluorescence proteins (DsREDs) from the non-bioluminescent coral Discosoma sp., mCherry, J-Red and mStrawberry; orange fluorescence protein (OFP) such as mOrange and mKO; cyan fluorescence protein (CFP) such as Cypet, mCFPm and Cerulean; and UV-excitable green fluorescence protein (UV-GFP) such as T-Sapphire. The stable markers in the cell lines of preferred embodiments of the present invention include, but are not limited to, the EGFP marker and firefly luciferase. However, those of skill in the art will recognize that modifications of the EGFP or luciferase protein other than that provided in commercially available EGFP or luciferase protein may be utilized in the practice of the present invention.

It is thus a second aspect of this invention to provide a cell, preferably a tumor cell, stably transfected with the expression composition of this invention. The expression composition described above may be introduced into tumor cells by routine techniques, which may vary and depend on the specific cell types or identity of the cells. For example, the transfection may be carried out using known reagent, such as “LIPOFECTAMINE™”. However, it will be readily understood by those of skill in the related art that other means of transfection may be utilized in the practice of this invention. Transfection may be accomplished by other lipid-type compounds, by chemical or electrical means, or by the use of viral vectors such as retrovirus. Examples of methods for introducing foreign nucleic acids into a cell include, but are not limited to, electroporation, cell fusion, DEAE-dextran mediated transfection, microinjection, proplast fusion, calcium phosphate-mediated transfection, lipofectine-mediated transfection, liposome delivery, particle bombardment and infection with a viral vector. Any suitable means of transfection may be utilized in the practice of the present invention.

In a preferred embodiment, the cells which were stably transfected with the expression composition of this invention were epithelial cells derived from human lung cancer. However, it will be readily understood by those of skill in the art that cells derived from other species and from other cell or tissue types may be stably transfected by the practice of this invention. For example, cells derived from other mammalian species may also be utilized in practicing this invention. Suitable cells include, but are not limited to, melanoma cells, ovary cancer cells, lung cancer cells, breast cancer cells and prostate cancer cells.

The present invention provides cell lines that are stably transfected with a to therapeutic protein and a visible marker. In one preferred example, epithelial cells derived from human lung cancer are modified to express cofilin and firefly luciferase. In another preferred example, epithelial cells derived from human lung cancer are modified to express cofilin and EGFP. The use of these cell lines makes possible of real time observation of biological events in vivo and in vitro. The cells may be monitored long-term without the need for histochemical or immunochemical treatment. However, it will be recognized by those of skill in the art that histochemical or immunochemical treatment may also be used in conjunction with the methods of the present invention. For example, if a second gene of interest is incorporated into the vectors of this invention, any appropriate means of detecting the presence of that gene may also be used in the practice of this invention.

According to preferred embodiments of this invention, cell lines that co-express cofilin and a visible marker (i.e., luciferase or EGFP) may be utilized as reagents for assessing tumor growth. As such, these cell lines may be provided either frozen or as live cells from an approved cell culture depository such as American Type Culture Collection (ATCC). Methods of culturing, freezing and shipping of cell lines are well known to those in the art.

The cell lines produced by the practice of this invention may be used in many lines of investigation in which it is desirable to monitor phenomena including, but not limiting to, cell migration, proliferation, apoptosis and/or cell shapes changes, in response to the expression of the candidate protein. For example, using cell lines of the present invention, it would be possible to monitor changes in cell growth in vivo or in vitro to correlate the changes with the expression of a particular protein of interest. The methods of the present invention would also provide the ability to insert the cell lines of this invention in an in vivo model (e.g., mouse), thereby allowing investigation of the expressed protein of interest on phenomena including, but not limiting to, tumor growth.

Thus, it is the third aspect of the present invention to provide a means for assessing therapeutic effects of a protein of interest on tumor growth while following the tumor simultaneously in vitro or in vivo. For in vitro application, tumor cells maintained in culture vessels were first treated with Dox, which may be accomplished by adding a suitable amount of Dox to the culture medium, so as to activate the tet-on system and thereby activating its downstream gene expression and express the candidate protein and the visible marker. The growth of the tumor cells in the culture vessels may then be followed in real time by monitoring the luminescent or fluorescent signal emitted from the expressed visible marker. If the expressed candidate protein possessed tumor growth inhibitory activity, the luminance or fluorescence intensity of the co-expressed marker would diminish with time. On the other hand, if the expressed candidate protein possessed minimal tumor growth inhibitory activity, then the luminance or fluorescence intensity would continue to increase with time. In the present disclosure, doxycyclin may be administered in an amount of about 1 to 1000 μg/Kg, preferably about 100 to 800 μg/Kg, more preferably about 400 to 600 μg/Kg, and most preferably about 500 μg/Kg. Suitable amount of Dox includes, but is not limited to, 1, 20, 50, 80, 100, 120, 150, 180, 200, 220, 250, 280, 300, 320, 350, 380, 400, 420, 450, 480, 500, 520, 550, 580, 600, 620, 650, 680, 700, 720, 750, 780, 800, 820, 850, 880, 900, 920, 950, 980 and 1000 μg/Kg.

Similarly, for in vivo application, the tumor cells, modified by the expression vectors of this invention, were administered, preferably by subcutaneously injection, to a suitable site of a subject (e.g., a non-human mammal), and the effects of the candidate protein on tumor growth may then be monitored in real time by the practice of this invention. In a preferred embodiment, doxycyclin was added to the drinking water and fed to the subject, who has been modified to contain a cell line of this invention. The cell line was followed in real time by monitoring the luminance or fluorescence intensity emitted from the co-expressed visible marker within the tumor cells. Again, if the expressed candidate protein possessed tumor growth inhibitory activity, the luminance or fluorescence intensity emitted form the co-expressed marker would diminish with time. On the other hand, if the expressed candidate protein possessed minimal tumor growth inhibitory activity, then the luminance or fluorescence intensity would continue to increase with time. Doxycyclin may be added to the drinking water in an amount from about 0.02 to 2 μg/ml, preferably from about 0.1 to 1.5 μg/ml, and more preferably in an amount of about 1 μg/ml. Suitable amount of Dox includes, but is not limited to, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 and 2.0 μg/ml.

It must be noted that as used herein and in the appended claims, the singular forms “a”, “an”, and “the” include plural referents unless the context clearly dictates otherwise.

Unless defined otherwise, all technical and scientific terms used herein have the same meaning as commonly understood by one of ordinary skill in the to art to which this invention belongs. Although any methods and materials similar or equivalent to those described herein can also be used in the practice of the present invention, exemplary methods and materials are described for illustrative purposes.

The following Examples are provided to illustrate certain aspects of the present invention and to aid those of skilled in the art in practicing this invention. These Examples are in no way to be considered to limit the scope of the invention in any manner.

### EXAMPLES

### Example 1

### Preparation of HCOXP-tk-Luc and HCOXP-TK-EGFP

### Cell Lines

### 1.1 Cell Culture

Human lung cancer H1299 epithelial cells were cultured in Dulbecco's modified Eagle's medium (DMEM) supplemented with 10% fetal calf serum (FCS), 2 mM L-glutamate, 50 μg/ml penicillin and streptomycin (Invitrogen, Carlsbad, Calif.). Cells were maintained in a humidified incubator at 37° C. with 5% CO₂ and 95% air, and were routinely passaged every two days.

### 1.2 Selection of HCOXP-tk Clones

Human cofilin cDNA (0.5 kb) was obtained by two-step reverse transcription-polymerase chain reaction (RT-PCR). Total RNA was extracted from H1299 cells using Trizol reagent (Invitrogen). For synthesis of the first strand cDNA, 1 mg of total RNA was used as template following the manufacturer's protocol of Superscript II (Invitrogen). The heterogeneous to first-strand cDNA was then subjected to polymerase chain reaction (PCR) using pfu polymerase (Stratagen, La Jolla, Calif.). Primers used for amplification of human cofilin cDNA were (1) 5′-CGCAGAATTCATGGCCTCCGGTGTGTGGCTGTC-3′ (SEQ ID NO: 1) and (2) 5′-GCGTGAATTCGCTCACAAAGGCTTGCCCTCC-3′ (SEQ ID NO: 2). PCR-amplified human cofilin cDNA was digested with EcoRI and inserted into the multiple cloning site of pBIG21 vector, which is an auto-regulated bi-directional tetracycline-inducible gene expression plasmid (see Strathdee et al., (1999) Gene 229, 21-29). Established constructs were stably transfected into H1299 cells of example 1.1 and were selected by hygromycin-B (0.2 mg/ml). The positive clone that over-expressed cofilin after doxycyclin (Sigma, St. Louis, Mo.) treatment was expanded as the HCOXP cell line, which was cultured and maintained in DMEM medium containing 10 μg/ml hygromycin-B and 10% serum in a humidified environment containing 95% air and 5% CO₂ at 37° C. 1.3 Selection of HCOXP-tk-Luc and HCOXP-tk-EGFP Clones

Two vectors were constructed; each vector contained a reporter gene (i.e., luciferase or EGFP) and herpes-simplex virus 1-thymidine kinase (HSV1-tk) gene. The two vectors were simultaneously induced by using a bidirectional tetracycline-inducible vector, and thereby producing pBI-Luc-HSV1-tk and pBI-EGFP-HSVi-tk, respectively. The original vector was pBI-Luc or pBI-EGFP purchased from Clontech Inc. HSV1-tk gene was in the pORF-HSV1-tk construct and was subcloned to pBI-Luc or pBI-EGFP vector through PvuII and NheI cloning sites. The vectors thus constructed were then used to transfect HCOXP-tk clones of Example 1.2, respectively, and the transfected cells were then selected by 1 μg/ml of puromycin to obtain stable cell lines. After amplification and transfer, HCOXP-tk-Luc clone was chosen for its high luminance and stability. Similarly, HCOXP-tk-EGFP clone was also chosen for its high fluorescence and stability.

### 1.4 Characterization of HCOXP-tk-Luc and HCOXP-tk-EGFP Clones of Example 1.3

HCOXP-tk-Luc and HCOXP-tk-EGFP Clones of Example 1.3 were respectively challenged with doxycyclin so as to verify the respective tet-on system in regulating the expression of cofilin and the reporter gene (i.e., luciferase of EGFP). The level of cofilin or thymidine kinase was measured by western blot analysis, whereas the expression of the reporter gene was confirmed by luciferase assay or fluorescence intensity measurement.

Western Blot Analysis Cells grown in 100-mm Petri dish were scraped and lysed in buffer containing 0.5% Igepal CA-630, 50 mM Tris-HCl, 120 mM NaCl and 1.5% phenylmethylsulphonyl fluoride (PMSF). Protein extracts were quantified by Bradford assay (Bio-Rad, Hercules, Calif.), For western blot assay, 70 μg of crude protein was loaded onto 15% sodium dodecyl sulfate-polyacrylamide gel and separated by sodium dodecyl sulfate-polyacrylamide gel electrophoresis (SDS-PAGE). The fractionated proteins were transferred to PVDF membrane and blocked with 4% skim milk in Tris-buffered saline with Tween-20 (TBST) buffer (0.8% NaCl, 0.02% KCl, 25 mM Tris-HCl/pH 8.0, and 0.1% Tween-20). The membrane was then incubated with various primary antibodies diluted in TBST containing 2% skim milk overnight at 4° C. After washing with TBST buffer, the membrane was incubated with horseradish peroxidase (HRP)-conjugated secondary antibody (1:5,000) for 2 to 3 hours at room temperature. Protein bands were detected using the ECL chemiluminescence reagent (Amersham Pharmacia Biotech) and visualized by exposure on X-ray film (Kodak, Rochester, N.Y.). The intensity of protein bands was measured by densitometry and quantified by NIH Image software. The primary antibodies used in this study were anti-cofilin polyclonal antibody, anti-TK (Santa Cruz, Calif.) and anti-β-actin monoclonal antibody (Sigma-Aldrich, St. Louis, Mo.).
Luciferase Assay Cells were grown in 6-well Petri dishes for at least 48 hours or until they reached about 80% confluence. The reporter assay buffer and lysed buffer were mixed and then added to each well, and the Petri dishes were then placed in a freezer at −80° C. for at least 10 minutes. The cells were then scraped out from the dishes and centrifuged for about 3 to 5 minutes. The clear lysate was collected and analyzed for luciferase activity by use of the luciferin containing assay buffer.

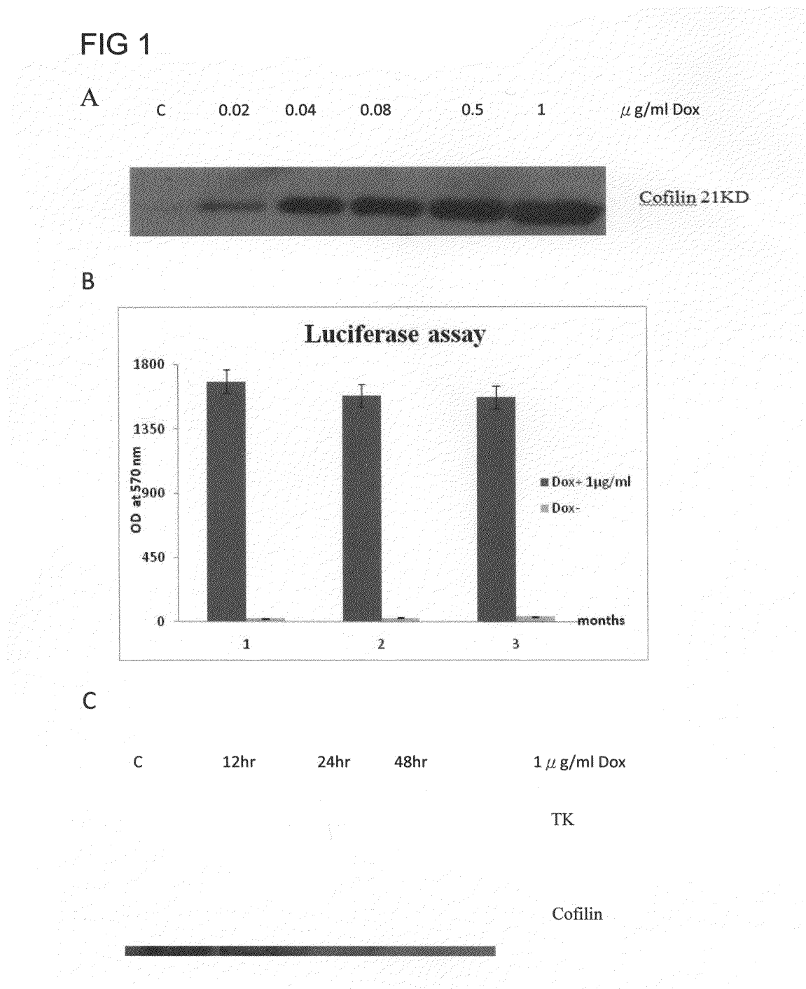

It is confirmed that the level of cofilin and luciferase activity of the selected HCOXP-tk-Luc clone of Example 1.3 are both regulated by the imbedded tet-on system, with the cofilin expression level and luciferase activity significantly increases along with an increase in doxycyclin concentration (FIGS. 1A and 1B). Similarly, the respective expression level of cofilin and thymidine kinase (TK) of the selected HCOXP-tk-EGFP clone of Example 1.3 are also regulated by the tet-on system, with the cofilin and TK expression levels significantly increase with time after doxycyclin treatment (FIG. 1C).

### Example 2

### Retarding and Monitoring Tumor Growth In Vivo by use of HCOXP-tk or HCOXP-tk-Luc Cell Line

### 2.1 Tumor Generation

To inoculate tumor in mice, mice were first anesthetized by 50 mg/ml ketamine and 150 mg/ml xylazine through i.p. injection. Then, 5×10⁶ HCOXP-tk cells of Example 1.2 and 5×10⁶ HCOXP-tk-Luc cells of Example 1.3 were injected subcutaneously to generate s.c. tumors at day 1. The mice with xenografted tumor cells (either HCOXP-tk cells of Example 1.2 or HCOXP-tk-Luc cells of Example 1.3) were randomly divided into two groups, and mice in each group were allowed to access food and water ad libitum, in which the drinking water was supplemented with or without doxycyclin (1 μg/ml). The tumor cells were allowed to grow for at least 5 weeks, and the size of tumor (mm³) was measured regularly (e.g., every 3 days) and calculated in accordance with the following equation:

TumorSize(mm³)=(length×width×width)/2

In which the length and width respectively represent the length of the tumor that is perpendicular or parallel to the hind legs of the mouse.

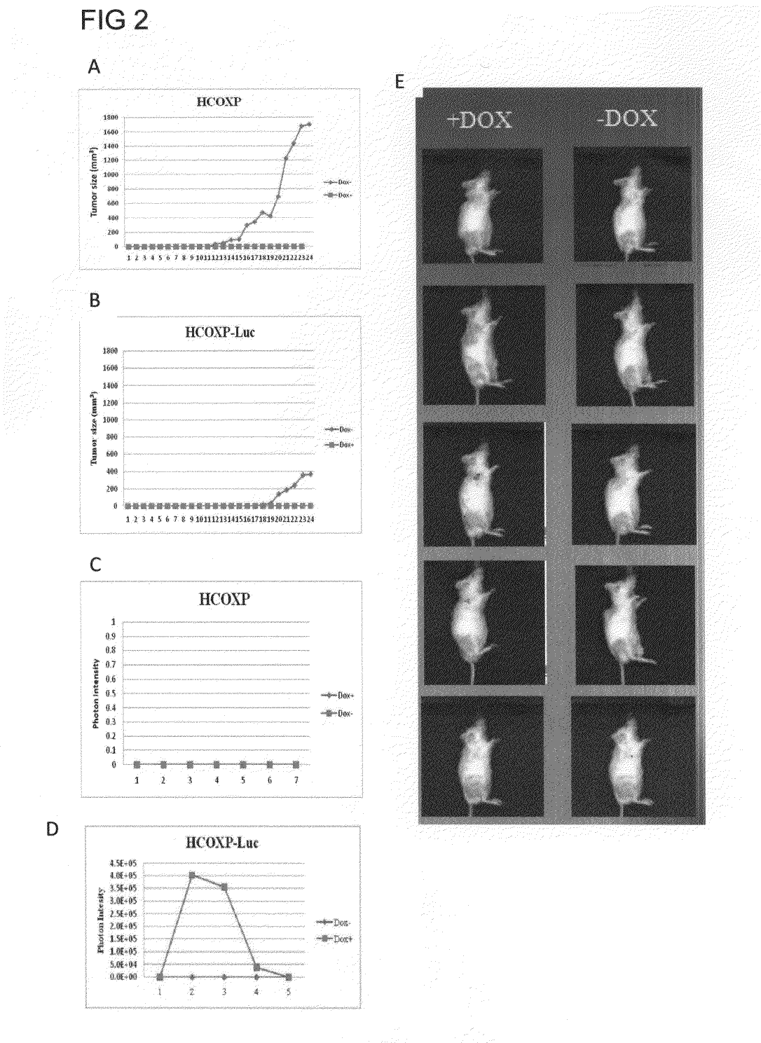

For mice inoculated with HCOXP-tk cells of example 1.2 or HCOXP-tk-luc cells of example 1.3, their tumor sizes increased significantly with time (FIGS. 2A and 2B, curves that indicate without Dox treatment), however, if mice were allowed to access drinking water supplemented with doxycyclin (1 μg/ml), which turned on the expression of cofilin and inhibited/retarded the growth of the tumors, as a result, tumor was barely detected after 24 days (FIGS. 2A and 2B, curves that indicate Dox treatment). As to luminance intensity, since the mice to having xenografted HCOXP-tk cells of example 1.2 would not express luciferase, hence none of the animals (with or without Dox treatment) exhibit detectable level of luminance (FIG. 2C), whereas mice having xenografted HCOXP-tk-Luc cells of example 1.3, luminance was high following inoculation of the tumor, then diminished with time as tumor cells began to express cofilin and thereby inhibited or suppressed the tumor growth (FIG. 2D).

### 2.2 In Vivo Monitoring the Xenografted Tumor of Example 2.1

The tumor was monitored in vivo by taking optical images of the mouse at regular time intervals by use of the In vitro imaging system (IVIS, Xenogen Corp, Alameda, Calif.). Briefly, the mice having xenografted HCOXP-tk-luc cells of example 1.3 were i.p. injected with D-luciferin (200 mg/Kg), and were subsequently anesthetized in a chamber filled with 2% isoflurane. After 15 minutes, each mouse was placed in the IVIS system with its body temperature maintained by a heating plate and scanned by IVIS system, with images being taken every minute. These images were further analyzed by suitable software.

FIG. 2E are IVIS images taken from mice having xenografted HCOXP-tk cells of example 1.2 and HCOXP-tk-luc cells of example 1.3 respectively in their left and right feet in the presence or absence of doxycyclin treatment. The images on the left panels of FIG. 2E were taken from a mouse, which had free access to doxycyclin (1 μg/ml) supplemented drinking water, whereas images on the right panels were taken from a mouse that had no access to doxycyclin (1 μg/ml) supplemented drinking water. Since both cofilin and luciferase were regulated by the tet-on system, hence, with the addition of doxycyclin, the expression of luciferase and cofilin would both be turned on, as a result, luminance was detected on the second week as tumor started to grow, at the same time, cofilin also accumulated within the tumor cells, which in turn suppressed the tumor growth, therefore, luminance signal started to diminish on week 4, so did the tumor size, by week 5, the luminance became undetectable (FIG. 2E, left panels), while the tumor size shrunk to an undetectable size.

### Example 3

### Retarding and Monitoring Tumor Growth In Vivo by Use of HCOXP-tk-Luc or HCOXP-tk-EGFP Cell Line

### 3.1 Monitoring Tumor Growth by Luminance or Fluorescence Measurement

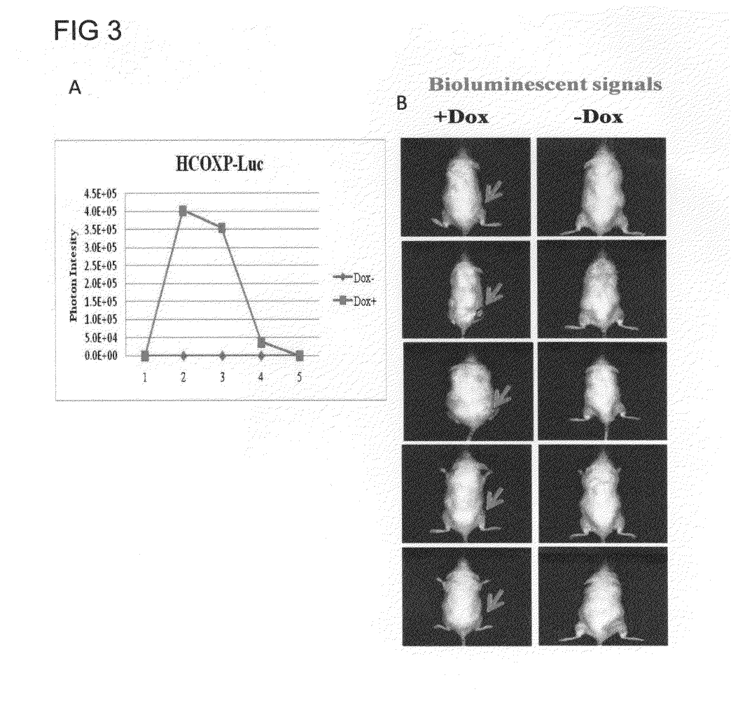

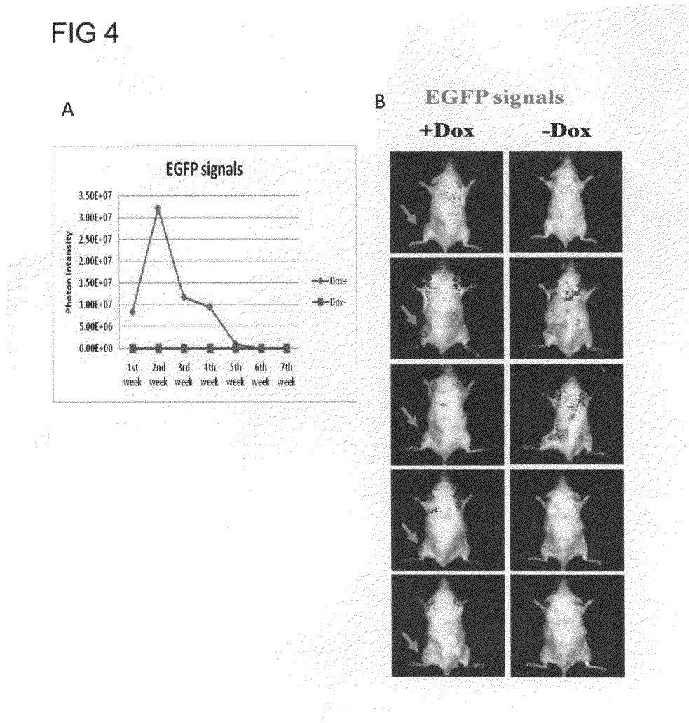

The mice were inoculated with 5×10⁶ HCOXP-tk-luc or HCOXP-tk-EGFP cells of Example 1.3 in accordance with the steps described in Example 2.1 to generate s.c. tumors. The growth of the tumor in each mouse was followed by measuring the luminance or fluorescence signal in vivo. Results were provided in FIGS. 3 and 4.

FIG. 3A illustrates the luminance intensity of the mouse having xenografted HCOXP-tk-luc cells in the presence or absence of doxycyclin treatment; and FIG. 3B are IVIS images taken from the same mouse from week 1 to 5, respectively, with the arrow indicated the site of the tumor. Luminance was measured in accordance with the procedures as described in Example 2.2. It was found that luminance reached its maximum on week 2, then, slowly diminished to an undetectable level by week 5 as the growth of tumor cells were inhibited by the over expressed cofilin. Similarly, for mice having xenografted HCOXP-tk-EGFP cells of Example 1.3, fluorescence intensity also peaked at the second week, then, quickly diminished to the base level by week 5 (FIG. 4). Fluorescence Intensity was measured by Nikon microscopy equipped with a Xenon lamp power supply and an EGFP filter set (OPTIMA G303 FL).

3.2 Monitoring the Expression of Cofilin and EGFP by microPET Imaging and Gamma Camera with Pinhole

In this example, microPET imaging and Gamma camera were used to confirm whether cofilin and thymidine kinase (tk) were indeed co-expressed in mice having xenografted HCOXP-tk-EGFP cells of Example 1.3.

For microPet imaging, animals having xenografted tumor cells were injected with ¹⁸F-2′-fluoro-2′-deoxy-1-β-D-arabionofuranosyl-5-ethyl-uracil (¹⁸F-FEAU), which served as the HSV1-tk substrate. One hour after i.v. administration of ¹⁸F-FEAU (50 μCi/animal), the mouse was anesthetized with 2% isoflurane and placed and fixed on the bed of the machine, then the whole body PET images were taken for 15 minutes. Radioactivity level within the tumor (% dose/g of tumor weight) was determined by software (ASI Pro 6.3.3) associated with the microPET (MicroPET Rodent R4, Simens).

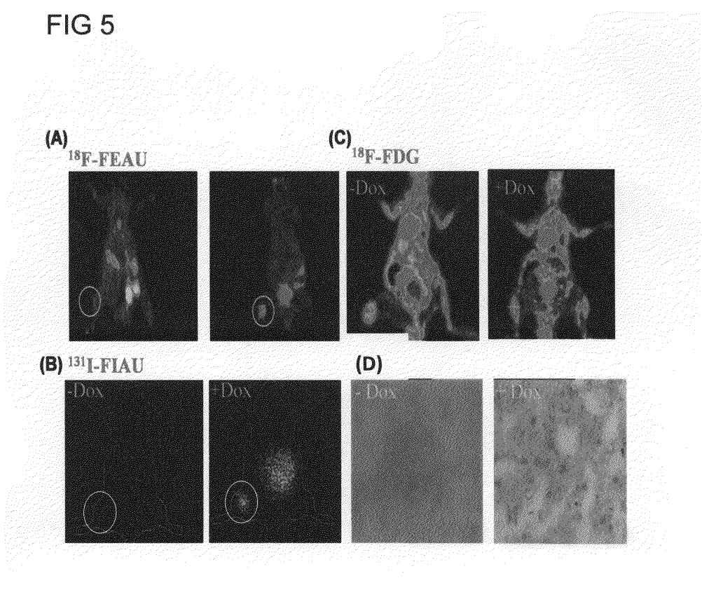

For gamma camera imaging, 100 μCi/0.1 ml [¹³¹I]-labeled 2′-fluoro-2′-deoxy-1-β-D-arabionofuranosyl-5-iodouracil (FIAU) was injected into mice that have been pretreated with 0.9% sodium iodine solution (Lugol's solution) to block the radioiodine uptake by the thyroid. After injection of [¹³¹I]-FIAU for at least 24 hours, the mouse was anesthetized with 2% isoflurane and placed and fixed on the bed of the gamma camera machine, then images were taken for 15 minutes. Results were provided in FIG. 5.

FIGS. 5A and 5B respectively illustrate the images taken by microPET and gamma camera from mouse having xenografted tumor cells with or without doxycyclin treatment. In FIGS. 5A and 5B, it was found that radioactivity within the region of interests (ROI, which is the circled area) in animals treated with to doxycyclin were higher than that without doxycyclin treatment, which confirmed that doxycyclin may effectively induce the co-expression of cofilin and thymidine kinase. In FIG. 5C, the ¹⁸F-FDG microPET image demonstrated the inhibitory activity showed in mice fed with water containing doxycycline, indicating cofilin expression. This observation was further confirmed by immunostaining of cofilin in ROI area (FIG. 5D).

Taken together, the model system of this invention as embodied in Examples herein indicates that the growth of a tumor may be retarded and tracked simultaneously in vivo by co-expressing cofilin and a light-emitting reporter gene respectively regulated by a tet-on system.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features. From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A composition, comprising:
a first vector comprises a first polynucleotide encoding a therapeutic protein candidate; and
a second vector comprises a second polynucleotide encoding a visible marker capable of emitting a luminescence or fluorescence signal;
wherein the first and second polynucleotides of the first and second vectors are respectively regulated by a tet-on system operably linked to the respective first and second polynucleotides for respectively regulating the to expression of the therapeutic protein candidate and the visible marker, such that the therapeutic protein candidate and the visible marker are simultaneously expressed upon activating the tet-on system.

2. The composition of claim 1, wherein the tet-on system is activated by doxycycline.

3. The composition of claim 1, wherein the first and the second vectors are respectively selected from the group consisting of an adenovirus, an adenovirus associated virus, a retrovirus and a lentivirus.

4. The composition of claim 3, wherein the first and second vectors are respectively replicable in normal or cancerous cells.

5. The composition of claim 1, wherein the visible marker is selected from the group consisting of luciferase, green fluorescence protein (GFP), yellow fluorescence protein (YFP), red fluorescence protein (RFP), orange fluorescence protein (OFP), cyan fluorescence protein (CFP), and UV-excitable green fluorescence protein (UV-GFP).

6. The composition of claim 5, wherein the visible marker is luciferase.

7. The composition of claim 5, wherein the visible marker is an enhanced green fluorescence protein (EGFP).

8. The composition of claim 1, wherein the therapeutic protein candidate is cofilin.

9. A cell stably transfected with the composition of claim 1.

10. The cell of claim 9, wherein the tet-on system is activated by doxycyclin.

11. The cell of claim 9, wherein the cell is a tumor cell that is selected from the group consisting of melanoma cell, ovary cancer cell, lung cancer cell, breast cancer cell and prostate cancer cell.

12. The cell of claim 11, wherein the cell is tracked by luminescence or fluorescence signal emitted from the visible marker.

13. A method for evaluating the therapeutic effects of a therapeutic protein candidate on a tumor while simultaneously following the growth of the tumor in a live subject, comprising:
injecting the subject with the cell of claim 9 or 11;
administering to the subject an effective amount of doxycyclin to activate the tet-on system; and
monitoring the growth of the tumor by the luminescence or fluorescence signal emitted from the visible marker;
wherein the luminescence or fluorescence signal would first increase with the growth of the tumor, and subsequently diminish if the growth of the tumor is retarded by the expressed therapeutic protein candidate.

14. The method of claim 13, wherein doxycyclin is administered to the subject in an amount of about 1 to 1000 μg/Kg.

15. The method of claim 13, wherein the first vector and the second vectors are respectively selected from the group consisting of an adenovirus, an adenovirus associated virus, a retrovirus and a lentivirus.

16. The method of claim 13, wherein the visible marker is selected from the group consisting of luciferase, green fluorescence protein (GFP), yellow fluorescence protein (YFP), red fluorescence protein (RFP), orange fluorescence protein (OFP), cyan fluorescence protein (CFP), and UV-excitable green fluorescence protein (UV-GFP).

17. The method of claim 13, wherein the therapeutic protein candidate is cofilin.

18. The method of claim 13, wherein the subject is a non-human mammal.


SEQUENCE LISTING (2 sequences)

SEQ ID NO: 1 - DNA, 33 bp, Artificial Sequence
  Completely synthetic
cgcagaattcatggcctccggtgtgtggctgtc

SEQ ID NO: 2 - DNA, 31 bp, Artificial Sequence
  Completely synthetic
gcgtgaattcgctcacaaaggcttgccctcc